(19) United States
(12) Patent Application Publication
(10) Pub. No.: US 2022/0339408 A1
(43) Pub. Date: Oct. 27, 2022

(54) MEDICAL ORGANIZATION APPARATUS

(71) Applicant: BE Innovative Medical Solutions, LLC, Denver, CO (US)

(72) Inventors: Michael Ryan Ballard, Denver, CO (US); Aaron Treat, Denver, CO (US); Maxavier M. Guss, Centennial, CO (US)

(21) Appl. No.: 17/660,283

(22) Filed: Apr. 22, 2022

Related U.S. Application Data
(60) Provisional application No. 63/178,375, filed on Apr. 22, 2021.

(51) Int. Cl. A61M 25/02

(52) U.S. Cl. CPC A61M 25/02; A61M 2025/024

(57) ABSTRACT

Medical apparatuses and medical systems may implement a channel component according to one or more aspects of the present disclosure. A channel component may include channels (e.g., a sequence or row of channels formed by base components and top components), such that cords and tubes may be inserted through the channels and efficiently organized/managed by medical professionals. For instance, an apparatus may include at least a base component, a top component, and a sheath component. The sheath component may be secured over tubes and cords of varying sizes, and the sheath component may then be secured between the base component and the top component. In some examples, multiple base components may be connected together, providing possible configuration of multiple channels when combined with additional top components/sheath components.

### CROSS-REFERENCE TO RELATED APPLICATION

This application claims priority from Provisional Patent Application No. 63/178,375 filed on Apr. 22, 2021, in the United State Patent and Trademark Office, and all the benefits accruing therefrom under 35 U.S.C. 119, the contents of which in their entirety are herein incorporated by reference.

### BACKGROUND

The following relates generally to medical procedures, and more specifically to securing medical components.

Medical procedures such as medical exams, surgical procedures, etc. may be performed using various types of specialized equipment. For instance, for performing various medical procedures, medical offices or operating rooms may include medical instruments or medical equipment such as scalpels, forceps, needles/syringes, spreaders, clamps, saws, rods, screws, stents, catheters, Intravenous (IV) tubes, feeding tubes, inhalational anesthetic tubes, sterilized lubricants, instrument sterilization trays, and topical cleaning solutions, among many other examples.

In addition to various types of specialized equipment, medical professionals may utilize patient beds or patient tables, medical equipment tables, machine racks, medical drapery, etc. in order to facilitate both efficient and sterile medical procedures. For example, in some cases, a surgical table may be located proximate to a patient area (e.g., an area or region of a patient being operated on, such as the brain, spine, heart, etc.) in order to facilitate quick access to necessary medical equipment during a medical procedure. Further, some medical procedures may be performed such that work surfaces of the surgical table, as well as the medical instruments to be used for the medical procedure, are sterilized prior to use (e.g., and kept sterile until used for the medical procedure).

However, due to the extensive amount medical equipment that may be involved in a given medical procedure, medical professionals may face challenges in managing the medical equipment during the procedure while maintaining an appropriately sterile environment. Improved techniques and devices for managing medical equipment may thus be desired.

### SUMMARY

An apparatus, system, and method for securing medical components are described. One or more aspects of the apparatus, system, and method include a base component having a base opening and a base attachment part; a top component having a top opening and a top attachment part configured to detachably connect to the base attachment part; and a sheath component having a first surface with a first complementary shape that fits within the top opening and a second surface with a second complementary shape that fits within the base opening, wherein the sheath component is configured to be removably inserted between the top component and the base component.

An apparatus, system, and method for securing medical components are described. One or more aspects of the apparatus, system, and method include a plurality of base components, wherein each of the plurality of base components has a base attachment part; a plurality of top components corresponding to the plurality of base components, wherein each of the plurality of top components has a top opening and a top attachment part configured to detachably connect to a corresponding base attachment part; and a plurality of sheath components, wherein each of the plurality of sheath components has an outer surface having a same shape as the top opening of a corresponding top component and is configured to be removably inserted into a corresponding top component.

A method for securing medical components is described. One or more aspects of the method include providing a base component having a base opening and a base attachment part; providing a top component having a top opening and a top attachment part configured to detachably connect to the base attachment part; and providing a sheath component having a first surface with a first complementary shape that fits within the top opening and a second surface with a second complementary shape that fits within the base opening, wherein the sheath component is configured to be removably inserted between the top component and the base component.

### BRIEF DESCRIPTION OF THE DRAWINGS

FIG. 1 shows a planer view of an example of system for securing medical components according to aspects of the present disclosure.

FIG. 2 shows a side view of an example of system for securing medical components according to aspects of the present disclosure.

FIG. 3 shows an example configuration of an apparatus according to aspects of the present disclosure.

FIG. 4 shows example base components of an apparatus according to aspects of the present disclosure.

FIG. 5 shows an example configuration of an apparatus according to aspects of the present disclosure.

FIG. 6 shows a side view of an example configuration of an apparatus according to aspects of the present disclosure.

FIGS. 7 through 8 show examples of a system for securing medical components according to aspects of the present disclosure.

FIG. 9 shows a side view of a sheath component inserted into a top component according to aspects of the present disclosure.

FIGS. 10 through 11 show examples of a system for securing medical components according to aspects of the present disclosure.

FIGS. 12 through 13 show examples of methods for medical procedures according to aspects of the present disclosure.

### DETAILED DESCRIPTION

The present disclosure relates to medical procedures. More specifically, the present disclosure may provide techniques and apparatuses that may be used to stabilize, organize, and support surgical accessories for various surgical procedures.

Medical procedures may demand organization of a surgical environment, as well as routine sterilization of a surgical environment (e.g., of medical instruments, medical tables, and various other medical equipment). In some cases (e.g., because surgical platforms and other medical tables may be large and difficult to sterilize), sterilization demands may be met via utilization of medical drapery. Medical drapery (e.g., sterilized disposable drapes, sterilized covers, surgical drapes, etc.) may refer to a barrier material used during surgical procedures to prevent contact between surfaces of tools, tables, or a surgical subject. Further, medical instruments and other various other medical equipment may also need to be efficiently organized. For instance, medical instruments and other various other medical equipment may be organized for efficient identification and utilization by operating medical professionals, for cleanliness and/or safety requirements, etc.

Moreover, medical procedures may be associated with a large amount of equipment and important sterilization demands. For instance, a surgical procedure may be associated with extensive medical drapery in addition to various medical instruments, medical equipment, tubes, cords, etc. For example, various tube elements (e.g., which may generally refer to medical equipment tubes, electrical cords, etc.) may be components of various devices that may be used during a medical procedure, for example, to send an electrical signal, to transmit power, to deliver medicine or compounds to a patient, to provide irrigation from a patient, etc. For example, tube elements (e.g., cords, tubes, etc.) in a surgical setting may include intravenous (IV) drips, inhalational anesthetic tubes, pulse sensors, miniature saws, water pumping devices, and various other medical equipment. Accordingly, for some medical procedures, numerous cords or tubes may be attached to devices and set in place on a patient or used as medical instruments by medical professionals.

With such a large amount of medical equipment and supplies, apparatuses and systems for efficient organization are demanded (e.g., to avoid misuse of the medical equipment, to avoid desterilization of medical equipment, to avoid inefficient or extended medical procedures, etc.). For instance, throughout some medical procedures, cords or tubes may become entangled or disorganized, may dislodge or disrupt medical drapery, may result in disorganization of other medical instruments, may provide sterilization challenges, etc. As some medical procedures may involve numerous medical components (e.g., cords, tubes, instruments, tools, etc.) surrounding a surgical table, disorganized cords, tubes, etc. may increase patient risk of infection, may result in susceptibility to cord damage or tube damage, etc.

Techniques and apparatuses described herein may provide for improved organization and securement of medical components (e.g., such as medical drapery, cords and tubes, medical instruments, etc.). For instance, some embodiments of the present disclosure may include a two-part apparatus design with a platform component (e.g., an external surface) and a channel component. The apparatus may be implemented to stabilize medical drapery, manage various cords and tubes, maintain sterilization of medical instruments and a surgical environment, stabilize a patient (e.g., with a single device), etc.

As described in more detail herein, a channel component may include channels (e.g., a sequence or row of channels formed by base components and top components) such that cords and tubes may be inserted through the channels and efficiently organized/managed by medical professionals. For instance, an apparatus (e.g., a channel of a channel component in a system for securing medical components) may include a base component, a top component, and a sheath component. The sheath component may be secured over tubes and cords of varying sizes, and the sheath component may then be secured between a base component and a top component (e.g., in a channel of a channel component). In some examples, multiple base components may be connected together, providing possible configuration of multiple channels when combined with additional top components/sheath components. Multiple tube elements may thus be secured via multiple channels of a channel component, and the channel component may then be attached to, or placed on, a platform component. Accordingly, various tube elements (e.g., medical tubes, cords, etc.) may be secured in place via the channels to prevent cords or tubes from becoming dislodged. As such, cords and tubes may flow to the patient in an organized fashion.

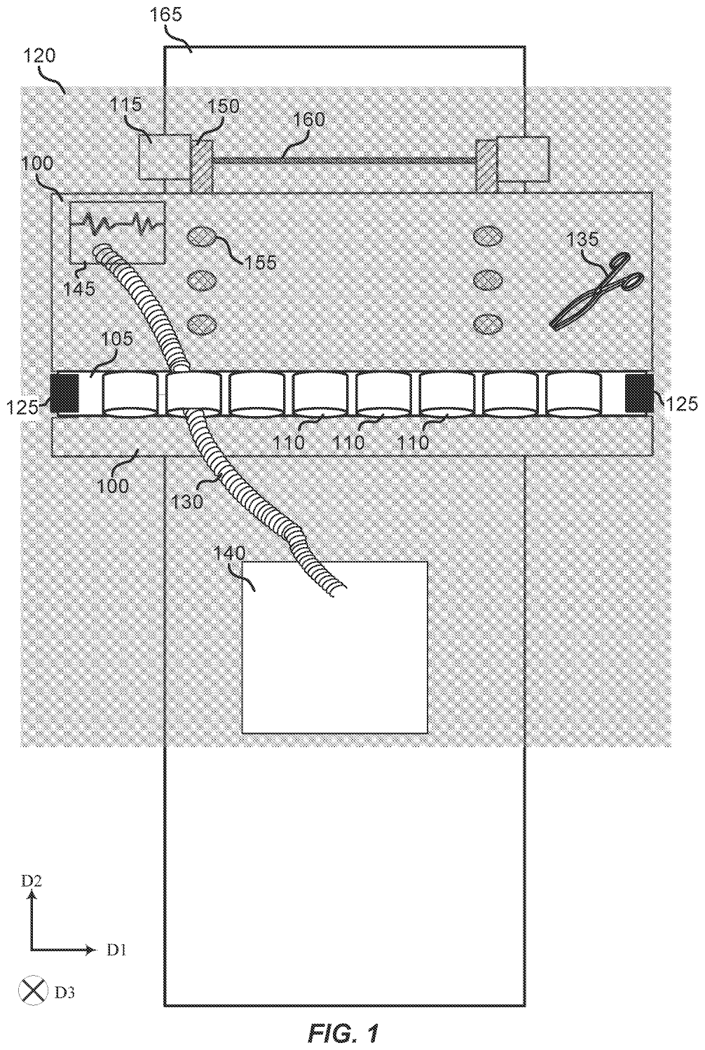

FIG. 1 shows an example of system for securing medical components according to aspects of the present disclosure. The example shown includes platform 100, channel component 105, channels 110, frame 115, medical drapery 120, attachment pieces 125, tube elements 130, tools 135, patient area 140, a medical machine 145, support arms 150, attachment holes 155, support rod 160, and medical table 165.

According to some embodiments, platform 100 may refer to a platform (e.g., a medical table, a surgical table, a surgical tray, etc.) with a flat surface. In some examples, the platform 100 includes one or more notches (e.g., notches 420, notches 515), and the channel component 105 may be attached to the platform 100 via attachment pieces 125 aligned with the notches with the medical drapery 120 therebetween. In some examples, the platform 100 includes one or more peg holes, and the channel component 105 includes one or more pegs (e.g., pegs 810) corresponding to the one or more peg holes, where the peg holes are configured to receive the pegs with the medical drapery 120 therebetween.

According to some embodiments, platform 100 may refer to a platform (e.g., a medical table, a surgical table, a surgical tray, etc.) with a flat surface configured to support a medical drapery 120. In some examples, the platform 100 is configured to be movable in a third direction (D3) while the attached channel component 105 secures the at least one tube element 130 during the medical procedure, the third direction (D3) being substantially perpendicular to a plane formed by the first direction (D1) and the second direction (D2). In some examples, the platform 100 is configured to support one or more surgical tools 135 in addition to the attached channel component 105.

According to some embodiments, channel component 105 comprises one or more channels 110, wherein each of the channels 110 is configured to admit and secure one or more tube elements 130 during a medical procedure. As described herein, the channel component 105 may be configured to attach to the platform 100 and secure a medical drapery 120 therebetween (e.g., via attachment pieces 125 and notches in the platform 100 and channel component 105, via peg holes in the platform and pegs of the channel component 105, or via various other mechanisms for attaching the channel component 105 to the platform 100). In some examples, each of the channels 110 includes a portion of a cylinder with an opening configured to receive the one or more tube elements 130.

In some examples, a tube element 130 may include an electrical cord, a bipolar cord, a suction tube, a water tube, a drill cord, a smoke evacuation cord, an electrocautery cord, an electrical stimulation cord, various other cords, or any combination thereof. In some examples, a tube element 130 may include an IV drip, feeding tubes, inhalational anesthetic tubes, various other tubes, or any combination thereof. Accordingly, as used herein, the terms “cord” and “tube” may generally be used interchangeably to refer to any components (e.g., medical components) securable by a channel component 105.

According to some embodiments, channel component 105 comprises a plurality of channels 110, wherein the channel component 105 is substantially the same size as the platform 100 in a first direction (D1) and is configured to attach to the platform 100 to secure the medical drapery 120 during a medical procedure. In some examples, the plurality of channels 110 is arranged in the first direction (D1) and is configured to admit and secure at least one tube element 130 during the medical procedure. In some examples, each of the channels 110 in the set of channels 110 includes a portion of a cylinder with an opening configured to receive one or more tube elements 130, the one or more tube elements 130 extending from the channel component 105 along a second direction (D2) substantially perpendicular to the first direction. In some examples, the channel component 105, the channels 110, and any channel locks (e.g., channel locks 215, channel locks 315, channel lock 1000) are sterilizable.

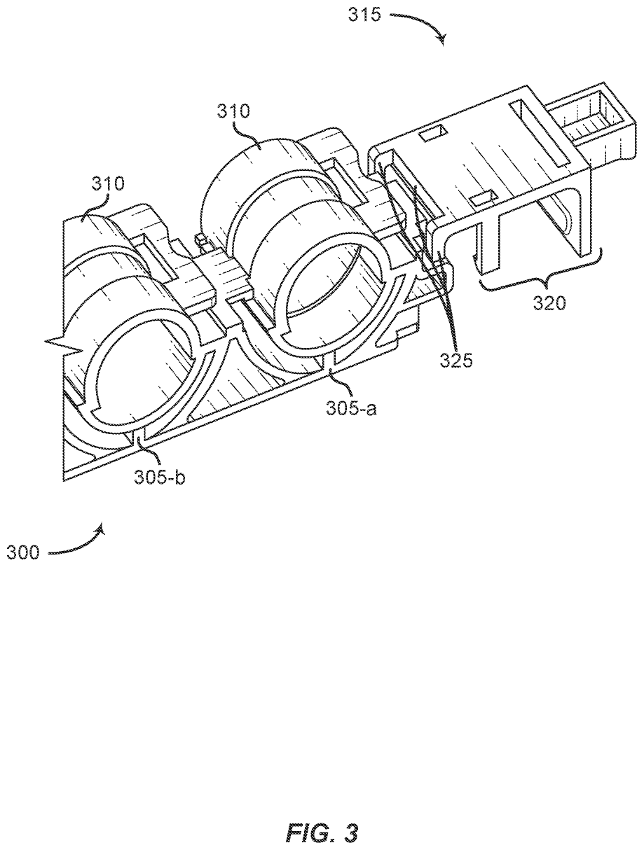

Channel component 105 may include one or more channels 110, and various channels 110 or combinations of channels 110 may further include a channel lock as described in more detail herein. As described in more detail herein (e.g., with reference to FIGS. 3-11), a channel 110 may include, or refer to, a base component. In some aspects, a channel 110 may include, or refer to, a base component and a top component. In some aspects, a channel 110 may further include, or refer to, a base component, a top component, and a sheath component.

According to some embodiments, attachment pieces 125 may include a top extension and a bottom extension and may be configured to secure one or more ends of the channel component 105 to one or more sides of the platform 100. In some examples, each of the one or more sides of the platform 100 comprises a groove (e.g., a notch) configured to receive the one or more attachment pieces 125, and the channel component 105 comprises an attachment notch configured to receive the top extension. In some examples, the bottom extension includes an extension peg, and the platform 100 includes an extension peg hole configured to receive the extension peg.

Attachment pieces 125 may generally include or refer to any attachment devices, clamps, hinged clamps, grips, brackets, fasteners, vices, etc. Tools 135 may generally include or refer to medical components such as scalpels, forceps, needles/syringes, spreaders, clamps, saws, rods, screws, or any other medical equipment.

In some cases, patient area 140 may refer to an area or region of a patient undergoing a medical procedure (e.g., such as a surgery). In some cases, patient area 140 may refer to an area set up by medical professionals as an area in which a surgical procedure may be performed. For instance, in some cases a patient area 140 may be formed such that a sterile environment surrounds the patient area 140 (e.g., where medical drapery 120 may surround the patient area 140, nearby medical components may be sterilized above medical drapery 120, medical components used for the medical procedure may be sterilized, etc.). Medical machines 145 may include or refer to any medical device or machine used for medical procedures. For instance, examples of a medical machine 145 may include an IV drip bag, a feeding machine, a pulse monitor, an anesthesia machine, etc.

According to some embodiments, support arms 150 are configured to attach the platform 100 to a frame 115 (e.g., a surgical bed frame). In some examples, the one or more support arms 150 may be permanently attached to the platform 100, and the channel component 105 may be separable from the platform 100.

According to some embodiments, support rods 160 may be configured to be inserted through first holes in the frame 115 (e.g., a surgical bed frame) and through second holes in the one or more support arms 150, thereby securing the apparatus to the frame 115. In some examples, a patient undergoing a medical procedure (e.g., surgery) may be positioned on a medical table 165. In some cases, medical table 165 may include or refer to a surgical bed. In some cases, frame 115 and medical table 165 may be a single component (e.g., a surgical bed frame, which may support a patient via a flat surface spanning a first and second direction, D1 and D2, and supply a mounting frame 115 in a third direction, D3)).

As further described herein, channel component 105 may be attached to the platform 100 in order to efficiently secure medical drapery 120 and facilitate sterilization of appropriate medical components (e.g., in addition to providing for improved organization of medical equipment, such as any of various tube elements 130 and tools 135 that may be used in a medical procedure). For instance, elements under medical drapery 120 (e.g., platform 100, various parts of frame 115 such as support arms 150, support rods 160, etc.). Alternatively, elements above medical drapery 120 (e.g., and in proximity of patient area 140) may be sterile or sterilized prior to, or during, a medical procedure. For instance, one or more medical machines 145 may be under medical drapery 120 (e.g., on platform 100 in a direction D2 away from the patient area, on the floor, etc.) and portions of one or more tube elements 130 attached to each of the medical machines 145 may be passed around the medical drapery 120 and through a channel 110 of the channel component 105. Accordingly, tube elements 130 may be sterilized and medical drapery 120 may be sterilized or disposed of after medical procedures without requiring the platform 100, the various medical machines 145, etc. be sterilized (e.g., as the medical drapery 120 may provide a barrier between non-sterilized elements or medical components and the patient area 140).

According to some embodiments, channel component 105 may include channels 110 (e.g., each channel 110 including a portion of cylinder with an opening configured to potentially receive a tube element 130) and channel locks (e.g., each channel lock configured to attach to one of the channels 110, thereby confining any tube elements 130 to the corresponding channel 110) which may all be readily sterilized. That is, channel component 105 (e.g., and all of channel component 105 elements or subcomponents) may be separable from platform 100 and may be sterilized between medical procedures (e.g., along with other medical components such as tools 135. Accordingly, the techniques and apparatuses described herein may provide for efficient securement of medical drapery 120, improved organization of tube elements 130 and other tools 135, and may facilitate medical procedures (e.g., via improved medical professional efficiency, surgical environment optimization, etc.) while satisfying sterilization demands.

In some cases, channel component 105 (e.g., and any elements, subcomponents, or pieces of a channel component 105) may be disposable, may be sterilized with other medical instruments (e.g., with any tools 135 used in a procedure), may be sterilized and pre-packaged or included in kits of surgical instruments, may be sterilized via sterilization trays or other sterilization receptacles, etc. Accordingly, channel component 105 may be suitable for securing medical drapery 120 and organizing medical components (e.g., such as tube elements 130).

Further, according to some embodiments, a sterile channel component 105 may provide for secure attachment to platform 100 (e.g., and thus to a rigid or sturdy frame 115 and/or medical table 165). As such, sterile channel component 105 may be utilized to stabilize a patient or one or more aspects of patient area 140 (e.g., in addition to one or more tube elements 130 or any other medical components). For instance, sterile channel component 105 may be suitable for stabilizing a patient's spine for surgical procedures, in addition to stabilizing one or more tubes or tube elements 130 for performing the surgical procedures.

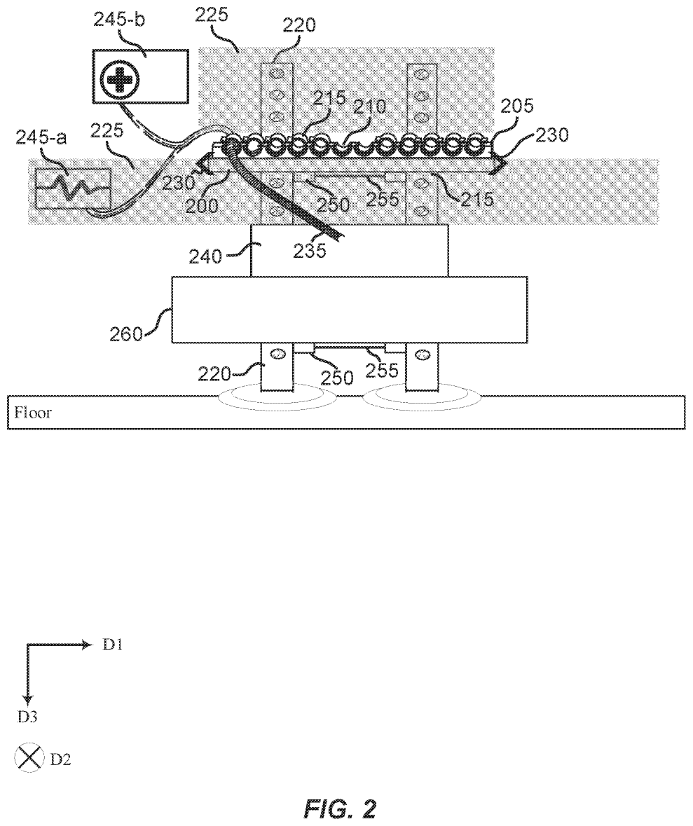

Platform 100, channels 110, frame 115, medical drapery 120, attachment pieces 125, patient area 140, medical machines 145, support arms 150, support rods 160, and medical table 165 are each examples of, or each include aspects of, the corresponding elements described with reference to FIG. 2. Channel component 105 is an example of, or includes aspects of, the corresponding element described with reference to FIGS. 2, 3, 5, 6, 10, and 11. Tube elements 130 are examples of, or include aspects of, the corresponding elements described with reference to FIGS. 2, and 7-11.

FIG. 2 shows an example of a system for securing medical components according to aspects of the present disclosure. The example shown includes platform 200, channel component 205, channels 210, channel locks 215, frame 220, medical drapery 225, attachment pieces 230, tube elements 235, patient area 240, medical machines 245, support arms 250, support rods 255, and medical table 260.

Medical apparatuses and medical systems may implement a channel component 205 according to one or more aspects of the present disclosure. A channel component 205 may include channels 210 (e.g., a sequence or row of channels 210 formed by base components and top components), such that cords and tubes may be inserted through the channels and efficiently organized/managed by medical professionals. For instance, an apparatus may include at least a base component, a top component, and a sheath component. The sheath component may be secured over tube elements 235 (e.g., tubes and cords) of varying sizes, and the sheath component may then be secured between the base component and the top component. In some examples, multiple base components may be connected together, providing possible configuration of multiple channels when combined with additional top components/sheath components. Accordingly, various tube elements 235 may be secured in place via the channels 210 to prevent tube elements 235 from becoming dislodged, to direct tube elements 235 to a patient area 240 or to one or more medical machines 245 in an organized fashion, etc.

In some examples, a patient may be positioned on a medical table 260. A patient area 240 may then be formed or established for performing a medical procedure (e.g., such as a surgical procedure) on the patient. For example, after the patient is positioned on the medical table 260, a platform 200 may be attached near the patient area 240 and medical drapery 225 may be placed over the platform 200. In some cases, the platform 200 may be attached to support arms 250 that may secure the platform 200 to a frame 220 (e.g., via two support rods 200) going through holes in the support arms 250 and the frame 220. Channel component 205 may then be attached to platform 200 to secure medical drapery 225 therebetween (e.g., between the platform 200 and the channel component 205). For instance, channel component 205 may be attached to platform 200 via attachment pieces 230.

As described herein, channel component 205 may include a plurality of channels 210 (e.g., such as parallel channels 210 that may be arranged in sequence along a first direction, D1). Further, channel component 205 may include channel locks 215 corresponding to any number of the plurality of channels 210 (e.g., channel component 205 may include zero channel locks 215, channel locks 215 for each channel 210 of a subset of the plurality of channels 210, or channel locks 215 for each channel 210 of all channels of the plurality of channels 210). As described, the channels 210 each include a portion of a cylinder with an opening configured to receive one or more tube elements 235 and the channel locks 215 each include a sliding portion. Further, each of the channels 210 include a groove configured to receive the sliding portion, and each of the channel locks 215 further include a notch configured to secure the channel lock 215 in a closed position. Accordingly, one or more tube elements 235 may be secured by each channel 210 and corresponding channel lock 215 without pinching, impeding, or damaging any of the secured tube elements 235. As an example, for some medical procedure applications, each channel 210 and corresponding channel lock 215 combination (e.g., when closed, when locked, when in a tube element 235 securing position, etc.) may have a diameter of 0.75 inches.

According to some embodiments, channel locks 215 may be configured to attach to one of the channels 210, thereby confining the tube element 235 to the corresponding channel. In some examples, each of the channel locks 215 includes a sliding portion, and each of the channels 210 includes a groove configured to receive the sliding portion, and where each of the channel locks 215 further includes a notch configured to secure the channel lock in a closed position. According to some embodiments, channel locks 215 may be configured to attach to at least one channel of the plurality of channels 210, thereby confining one or more tube elements 235 to the corresponding channel.

In some example, frame 200 and medical table 260 may be a single component (e.g., such as a surgical bed frame 220). In other examples, frame 200 may be a separate component from medical table 260, and the frame 220 may attach to the medical table 260 via support arms 250 attached to the medical table 260 (e.g., where one or more support rods 255 may attach the medical table 260 and the frame 220 through holes in each of the medical table 260 and the frame 220).

Various platforms 200 for securing medical components may be implemented in accordance with the described techniques without departing from the scope of the present disclosure. For instance, a platform 200 may be designed and implemented based on various factors such as the surgical environment (e.g., the size of the operating room), the medical table 260 and/or the frame 220 (e.g., the type or dimensions of the medical table 260 and/or the frame 220), the medical components being used (e.g., the types of medical instruments or the amount of medical instruments), etc. As an example, a platform 200 include a platform of 12 inches in a second direction D2 (e.g., in length) and 18 inches in a first direction D1 (e.g., in width), with various heights (e.g., with various dimensions in a third direction D3). As another example, a platform 200 include a platform of 24 inches in a second direction D2 (e.g., in length) and 18 inches in a first direction D1 (e.g., in width), with various heights (e.g., with various dimensions in a third direction D3). In some cases, the platform 200 may attach directly to (e.g., and have dimensions facilitating direct attachment to) the frame 220 used in the surgical setting (e.g., such as a Jackson frame).

In some cases, platform 200 may attach to frame 220 (e.g., which in some cases may refer to a metal piece with all the holes in it, such as an “H” bracket). For instance, frame 220 (e.g., a H bracket) may have 1 hole to accept a small post in the vertical direction, and a second hole to slide the metal cylinder pin through. Brackets of other platforms 200 (e.g., platforms 200 of varying dimensions) may be designed to fit on the bed (e.g., medical table 260) the same way the H bracket is designed to fit on, accept the brackets may travel horizontally instead or vertically. In some cases, the platform may use a single pin to attach to the bed.

Platform 200, channels 210, frame 220, medical drapery 225, attachment pieces 230, patient area 240, medical machines 245, support arms 250, support rods 255, and medical table 260 are each examples of, or each include aspects of, the corresponding elements described with reference to FIG. 1. Channel component 205 is an example of, or includes aspects of, the corresponding element described with reference to FIGS. 1, 3, 5, 6, 10, and 11. Tube elements 235 are examples of, or include aspects of, the corresponding elements described with reference to FIGS. 1, and 7-11.

FIG. 3 shows an example of a configuration of an apparatus according to aspects of the present disclosure. The example shown includes channel component 300 and coupling component 315. An apparatus may include a channel component 300 (e.g., which may be referred to as a Rotolock Modular Base, in some cases) and a coupling component 315 (e.g., which may be referred to as a hinge, in some cases). For example, a Rotolock Modular Base and Hinge may include 3 components that are used together to make an assembly.

In some aspects, the coupling component 315 may be removable (e.g., from the channel component 300) and may separate from a base component. In some examples, the coupling component 315 interacts with only the end base (e.g., base component 305-a) via a rotatory slot/channel system the secures the coupling component 315 when rotated but allows for easy installation and removal. In some examples, the coupling component 315 may be also secured with side snap hooks that attach to the end base component 305-a.

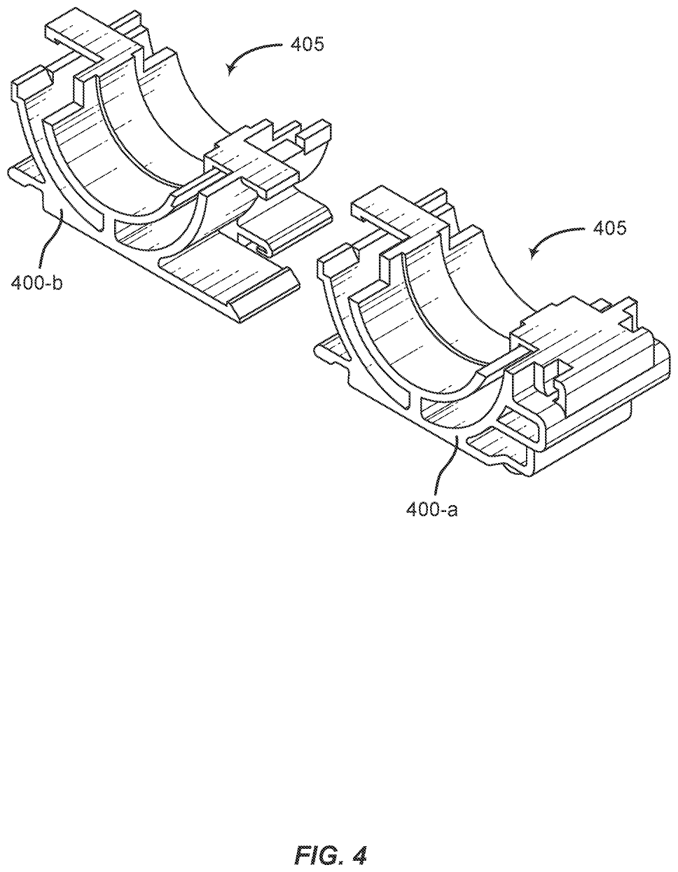

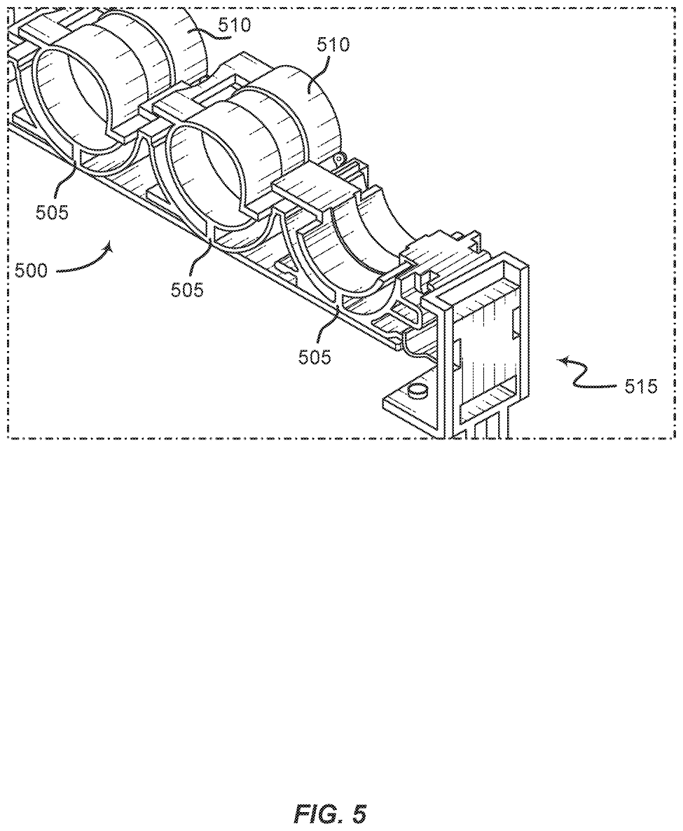

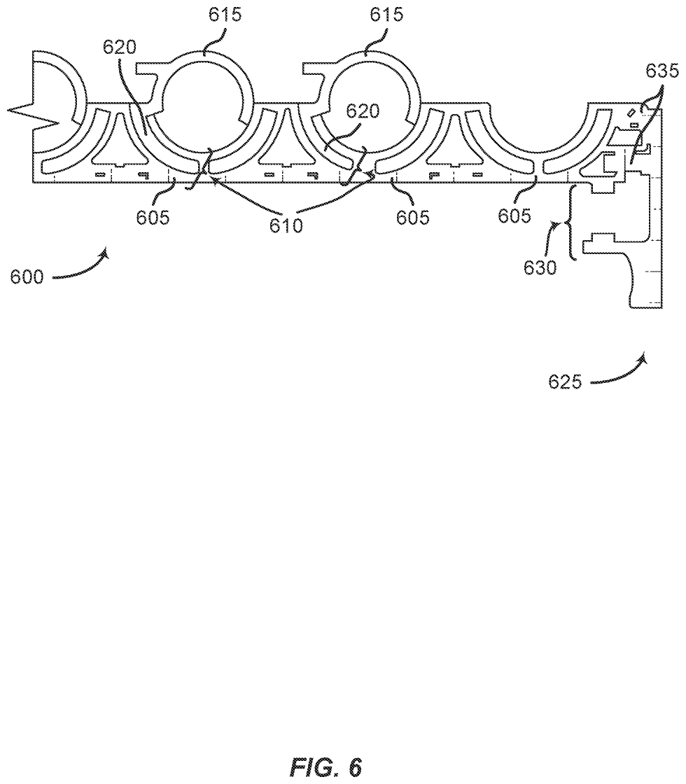

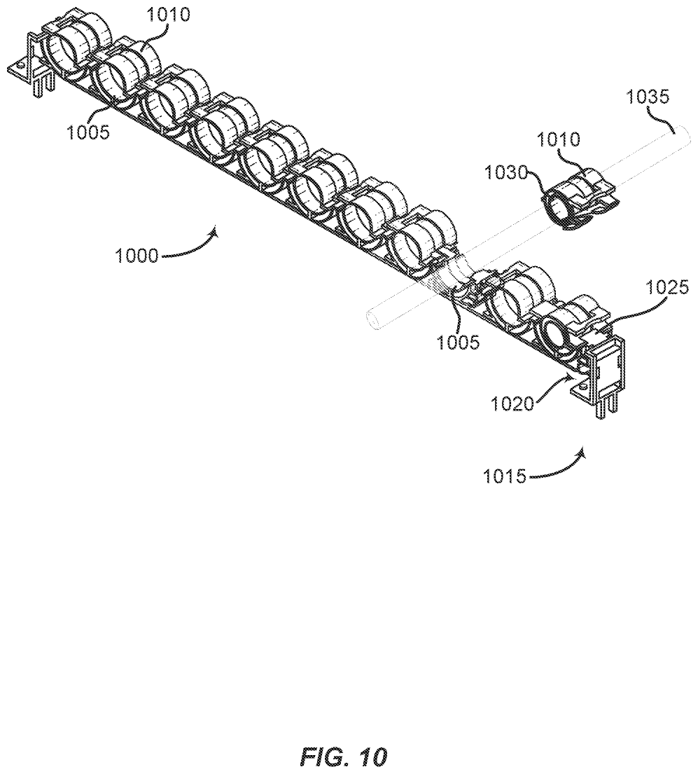

Channel component 300 is an example of, or includes aspects of, the corresponding element described with reference to FIGS. 1, 2, 5, 6, 10, and 11. In one aspect, channel component 300 includes base component 305 and top component 310. In some aspects, base component 305-a may be referred to as an end base component, and base component 305-b may be referred to as a segmental base component. Base component 305 is an example of, or includes aspects of, the corresponding element described with reference to FIGS. 4-6, 10, and 11. Top component 310 is an example of, or includes aspects of, the corresponding element described with reference to FIGS. 5, 6, and 8-11. Coupling component 315 is an example of, or includes aspects of, the corresponding element described with reference to FIGS. 5, 6, 10, and 11. In one aspect, coupling component 315 includes structure attachment part 320 and base attachment part 325. Structure attachment part 320 is an example of, or includes aspects of, the corresponding element described with reference to FIGS. 6 and 10. Base attachment part 325 is an example of, or includes aspects of, the corresponding element described with reference to FIGS. 6 and 10.

FIG. 4 shows an example of base components 400 of an apparatus according to aspects of the present disclosure. Base component 400 is an example of, or includes aspects of, the corresponding element described with reference to FIGS. 3, 5, 6, 10, and 11. In one aspect, base component 400 includes base opening 405.

For example, an end segment (e.g., a base component 400-a) may be joined to inner segments (e.g., inner base components 400-b, which in some examples may be referred to as segmental base components 400-b) a number of times to configure or produce a channel component (e.g., a Rotolock base assembly). For instance, end base component 400-a and segmental base component 400-b may interact through top and bottom snap hooks and multiple segmental base components 400-b can interact together to form assemblies (e.g., channel components) including two or more channels.

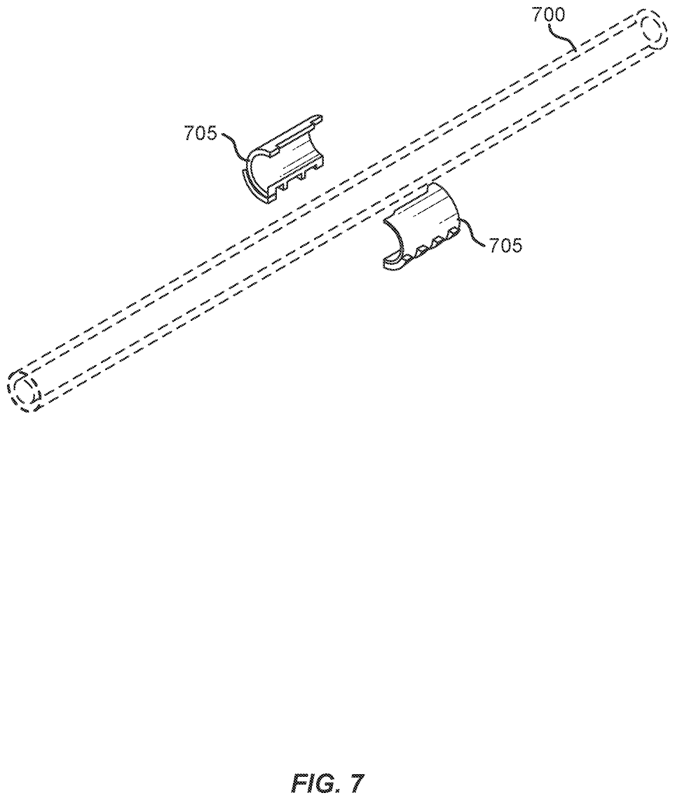

In some examples, a raised channel in the center of the base components 400 may interact with a separate sheath piece and top designed to secure medical devices, tubes, cords, or wires of various diameters (e.g., as described in more detail herein, for example, with reference to FIGS. 7-11).

FIG. 5 shows an example of a configuration of an apparatus according to aspects of the present disclosure. The example shown includes channel component 500 and coupling component 515.

The coupling component 515 may be inserted and rotated to secure the base assembly 505 (e.g., the end base assembly, the channel component 505, etc.) to an external surface it rests on (e.g., such as a platform described with reference to FIGS. 1 and 2, or to any other external surface). As an example, the coupling component may configure the channel component 500 to sit appropriately positioned on a platform when the raised channel on the underside of the base slides into the slot channel on the platform.

Channel component 500 is an example of, or includes aspects of, the corresponding element described with reference to FIGS. 1-3, 6, 10, and 11. In one aspect, channel component 500 includes base component 505 and top component 510. Base component 505 is an example of, or includes aspects of, the corresponding element described with reference to FIGS. 3, 4, 6, 10, and 11. Top component 510 is an example of, or includes aspects of, the corresponding element described with reference to FIGS. 3, 6, and 8-11. Coupling component 515 is an example of, or includes aspects of, the corresponding element described with reference to FIGS. 3, 6, 10, and 11.

FIG. 6 shows an example of a side view of a configuration of an apparatus according to aspects of the present disclosure. The example shown includes channel component 600 and coupling component 625.

Some examples of the apparatuses and systems described herein include a coupling component 625 connected to a base component 605, where the coupling component 625 includes a structure attachment part 630 configured to connect the coupling component 625 to an external structure and a base attachment part 610 configured to connect the coupling component 625 to the base component 605. As an example, the coupling component 625 may configure the channel component 600 to sit appropriately positioned on a platform when the raised channel (e.g., the structure attachment part 630) on the underside of the base slides into a slot channel on the platform.

In some aspects, the base attachment part 610 includes a snap hook that is detachable from the base component 605.

As described herein, the base component 605 has a base opening and a base attachment part 610, and the top component 615 has a top opening and a top attachment part 620 configured to detachably connect to the base attachment part 610. In some aspects, the top attachment part 620 includes an extension part that is configured to be inserted into a slot of the base attachment part 610 with a rotational motion of the top attachment part 620 around a central axis of the top opening.

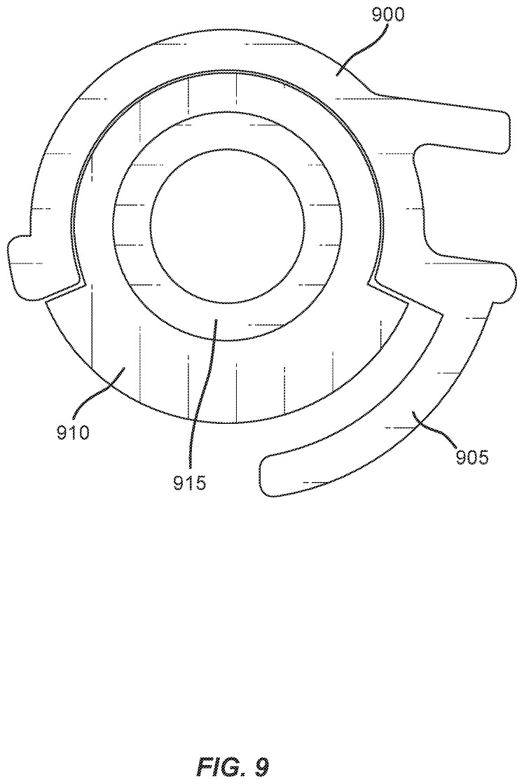

Channel component 600 is an example of, or includes aspects of, the corresponding element described with reference to FIGS. 1-3, 5, 10, and 11. In one aspect, channel component 600 includes base component 605 and top component 615. Base component 605 is an example of, or includes aspects of, the corresponding element described with reference to FIGS. 3-5, 10, and 11. In one aspect, base component 605 includes base attachment part 610. Base attachment part 610 is an example of, or includes aspects of, the corresponding element described with reference to FIGS. 3 and 10. Top component 615 is an example of, or includes aspects of, the corresponding element described with reference to FIGS. 3, 5, and 8-11. In one aspect, top component 615 includes top attachment part 620. Top attachment part 620 is an example of, or includes aspects of, the corresponding element described with reference to FIG. 9.

Coupling component 625 is an example of, or includes aspects of, the corresponding element described with reference to FIGS. 3, 5, 10, and 11. In one aspect, coupling component 625 includes structure attachment part 630 and base attachment part 610. Structure attachment part 630 is an example of, or includes aspects of, the corresponding element described with reference to FIGS. 3 and 10. Base attachment part 610 is an example of, or includes aspects of, the corresponding element described with reference to FIGS. 3 and 10.

FIG. 7 shows an example of aspects of a system for securing medical components according to aspects of the present disclosure. The example shown includes tube element 700 and sheath component 705. FIG. 7 shows one or more aspects of two halves of a sheath component 705 being snapped together around a tube element 700 (e.g., which may include a tube, cord, or medical device).

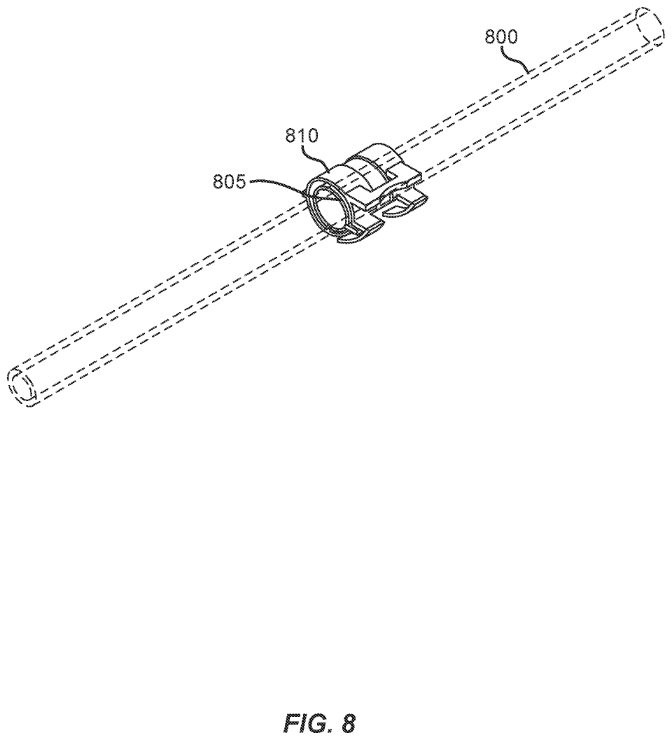

Apparatuses and systems described herein may include a sheath component 705 (e.g., as described in more detail herein, for example, with reference to FIGS. 8 through 11). In some aspects, the sheath component 705 has a circular opening configured to wrap around a tube element 700 (e.g., a medical tube, a cord, etc.) and affix the tube element to the apparatus. In some aspects, the sheath component 705 includes a first half-pipe part and a second half-pipe part that is detachably connected to the first half-pipe part.

Cords 700 is an example of, or includes aspects of, the corresponding element described with reference to FIGS. 1, 2, and 8-11. Sheath component 705 is an example of, or includes aspects of, the corresponding element described with reference to FIGS. 8-11.

FIG. 8 shows an example of aspects of a system for securing medical components according to aspects of the present disclosure. The example shown includes tube element 800, sheath component 805, and top component 810. FIG. 8 shows that, once around the tube element 800, the sheath component 805 may slide into a top component 810.

Cords 800 is an example of, or includes aspects of, the corresponding element described with reference to FIGS. 1, 2, 7, and 9-11. Sheath component 805 is an example of, or includes aspects of, the corresponding element described with reference to FIGS. 7, and 9-11. Top component 810 is an example of, or includes aspects of, the corresponding element described with reference to FIGS. 3, 5, and 9-11.

FIG. 9 shows an example of a side view of a sheath component 910 inserted into a top component 900 according to aspects of the present disclosure. The example shown includes top component 900, sheath component 910, and tube element 915. FIG. 9 shows that the shape of the sheath component 910 is configured as a similar shape (e.g., a shape configured to connect with) the shape of the inside of the top component 900).

Top component 900 is an example of, or includes aspects of, the corresponding element described with reference to FIGS. 3, 5, 6, 10, and 11. In one aspect, top component 900 includes top attachment part 905. Top attachment part 905 is an example of, or includes aspects of, the corresponding element described with reference to FIG. 6. Sheath component 910 is an example of, or includes aspects of, the corresponding element described with reference to FIGS. 7, 8, 10, and 11. Cords 915 is an example of, or includes aspects of, the corresponding element described with reference to FIGS. 1, 2, 7, 8, 10, and 11.

FIG. 10 shows an example of aspects of a system for securing medical components according to aspects of the present disclosure. The example shown includes channel component 1000, coupling component 1015, sheath component 1030, and tube element 1035.

An apparatus and system for securing medical components is described. One or more aspects of the apparatus include a base component 1005, a top component 1010, and a sheath component 1030. In some aspects, the base component 1005 has a base opening and a base attachment part 1025, and the top component 1010 has a top opening and a top attachment part configured to detachably connect to the base attachment part 1025. In some aspects, the sheath component 1030 has a first surface with a first complementary shape that fits within the top opening of the top component 1010 and a second surface with a second complementary shape that fits within the base opening of the base component 1005 (e.g., such that the sheath component 1030 is configured to be removably inserted between the top component 1010 and the base component 1005).

Some examples of the apparatus and system further include a set of additional base components 1005, where one of the set of additional base components 1005 is detachably connected to the base component 1005 and each of the set of additional base components 1005 is detachably connected to another of the set of additional base components 1005.

Some examples of the apparatus and system further include a set of additional top components 1010 and a set of additional sheath components 1030, where each of the set of additional top components 1010 and each of the set of additional sheath components 1030 corresponds to an additional base component 1005 of the set of additional base components 1005. In some aspects, each of the set of additional sheath components 1030 includes a sheath opening having a same size and shape as the sheath component 1030. In some aspects, at least one of the set of additional sheath components 1030 includes a sheath opening having a different shape from the sheath component 1030.

In some aspects, the base component 1005 includes an upper surface having a half-pipe shape corresponding to a bottom surface of the sheath component 1030.

In some aspects, the base component 1005, the top component 1010, and the sheath component 1030 are formed of a same material. In some aspects, the same material includes a rigid plastic material.

As described herein, in some examples, a base component 1005 may be connected to a coupling component 1015, where the coupling component 1015 includes a structure attachment part 1020 and a base attachment part 1025. The structure attachment part 1020 may be configured to extend around an edge of an external structure to fix a position of the coupling component 1015 relative to the external structure. The base attachment part 1025 may be configured to fix movement of the base component 1005 in a first direction and allow radial movement of the base component 1005 around the base attachment part 1025. In some examples, a second base component 1005 (e.g., a base component 1005 on an opposite end of a channel component 1000) may be attached to a second coupling component 1015, where the second coupling component 1015 is configured to connect to the external structure to restrict the radial movement of the base component 1005.

Channel component 1000 is an example of, or includes aspects of, the corresponding element described with reference to FIGS. 1-3, 5, 6, and 11. In one aspect, channel component 1000 includes base component 1005 and top component 1010. Base component 1005 is an example of, or includes aspects of, the corresponding element described with reference to FIGS. 3-6, and 11. Top component 1010 is an example of, or includes aspects of, the corresponding element described with reference to FIGS. 3, 5, 6, 8, 9, and 11. Coupling component 1015 is an example of, or includes aspects of, the corresponding element described with reference to FIGS. 3, 5, 6, and 11. In one aspect, coupling component 1015 includes structure attachment part 1020 and base attachment part 1025. Structure attachment part 1020 is an example of, or includes aspects of, the corresponding element described with reference to FIGS. 3 and 6. Base attachment part 1025 is an example of, or includes aspects of, the corresponding element described with reference to FIGS. 3 and 6. Sheath component 1030 is an example of, or includes aspects of, the corresponding element described with reference to FIGS. 7-9, and 11. Cords 1035 is an example of, or includes aspects of, the corresponding element described with reference to FIGS. 1, 2, 7-9, and 11.

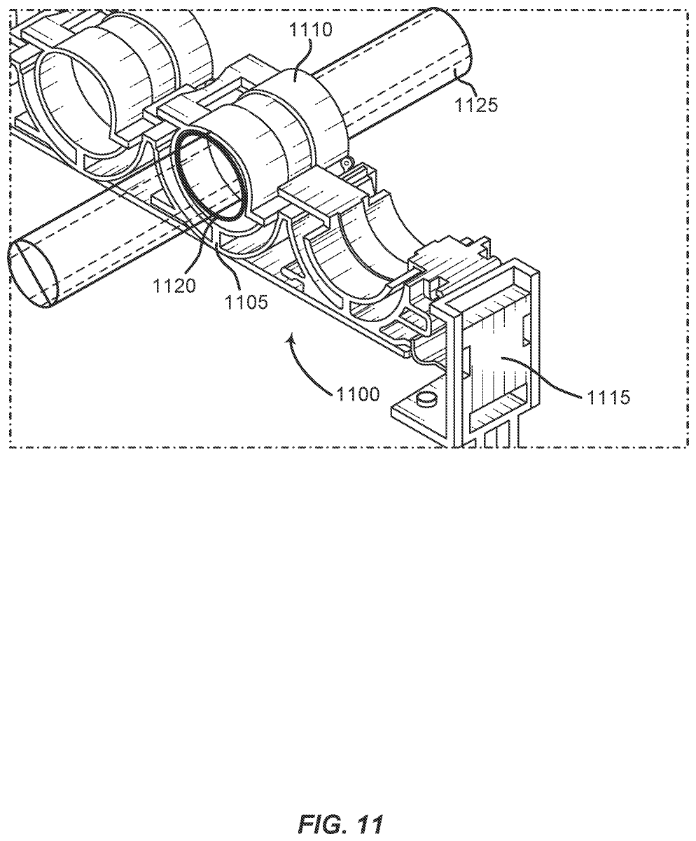

FIG. 11 shows an example of aspects of a system for securing medical components according to aspects of the present disclosure. The example shown includes channel component 1100, coupling component 1115, sheath component 1120, and tube element 1125. In some aspects, FIG. 11 shows that once the sheath component 1120 and the top component 1110 are connected, the top component 1110 may be inserted into the base (e.g., into the channel component 1100, into the base component 1105, etc.) by rotating it into the base component 1105. In some examples, a raised channel in the middle of the base component 1105 may interact with the slot on the top component 1110 and may align the two pieces to allow for smooth rotation and locking into place securely.

An apparatus and system for securing medical components is described. One or more aspects of the apparatus include a set of base components 1105, where each of the set of base components 1105 has a base attachment part; a set of top components 1110 corresponding to the set of base components 1105, where each of the set of top components 1110 has a top opening and a top attachment part configured to detachably connect to a corresponding base attachment part; and a set of sheath components 1120, where each of the set of sheath components 1120 has an outer surface having a same shape as the top opening of a corresponding top component 1110 and is configured to be removably inserted into a corresponding top component 1110.

Some examples of the apparatus and system further include a coupling component 1115 connected to a base component 1105 of the set of base components 1105, where the coupling component 1115 includes a structure attachment part configured to fix a position of the coupling component 1115 relative to an external structure and a base attachment part configured to fix movement of the set of base components 1105 in a first direction and allow movement of the set of base components 1105 in a radial motion around the base attachment part.

In some aspects, each of the set of base components 1105 includes a bottom surface that is parallel to a bottom surface of each of the other base components 1105 of the set of base components 1105.

Channel component 1100 is an example of, or includes aspects of, the corresponding element described with reference to FIGS. 1-3, 5, 6, and 10. In one aspect, channel component 1100 includes base component 1105 and top component 1110. Base component 1105 is an example of, or includes aspects of, the corresponding element described with reference to FIGS. 3-6, and 10. Top component 1110 is an example of, or includes aspects of, the corresponding element described with reference to FIGS. 3, 5, 6, and 8-10. Coupling component 1115 is an example of, or includes aspects of, the corresponding element described with reference to FIGS. 3, 5, 6, and 10. Sheath component 1120 is an example of, or includes aspects of, the corresponding element described with reference to FIGS. 7-10. Cords 1125 is an example of, or includes aspects of, the corresponding element described with reference to FIGS. 1, 2, and 7-10.

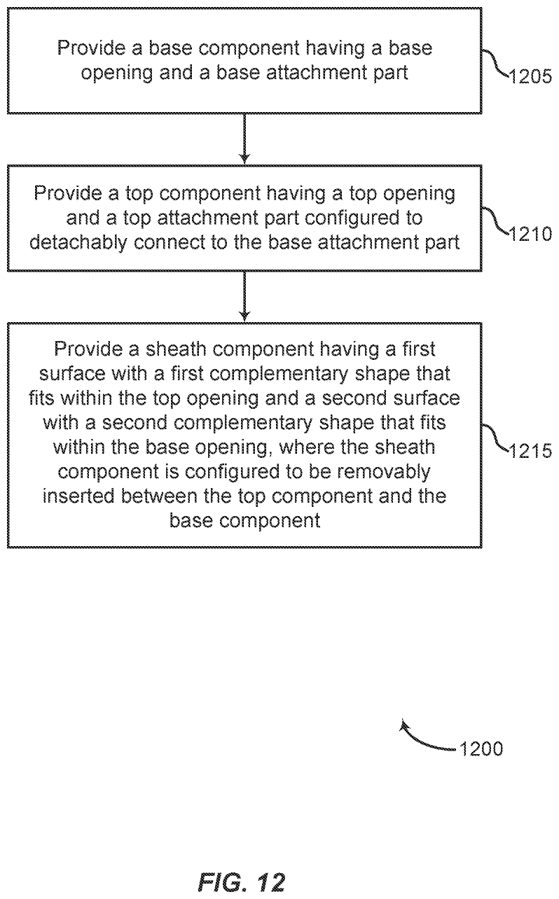

FIG. 12 shows an example of a method 1200 for medical procedures according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

In some cases, various aspects of operations 1205-1215 may be performed by, or facilitated by a medical professional (e.g., as described in more detail herein).

At operation 1205, the system provides a base component having a base opening and a base attachment part. In some cases, the operations of this step refer to, or may be performed by, a base component as described with reference to FIGS. 3-6, 10, and 11.

At operation 1210, the system provides a top component having a top opening and a top attachment part configured to detachably connect to the base attachment part. In some cases, the operations of this step refer to, or may be performed by, a top component as described with reference to FIGS. 3, 5, 6, and 8-11.

At operation 1215, the system provides a sheath component having a first surface with a first complementary shape that fits within the top opening and a second surface with a second complementary shape that fits within the base opening, where the sheath component is configured to be removably inserted between the top component and the base component. In some cases, the operations of this step refer to, or may be performed by, a sheath component as described with reference to FIGS. 7-11.

Some examples of the method further include attaching the base component to a coupling component, where the coupling component includes a structure attachment part configured to extend around an edge of an external structure to fix a position of the coupling component relative to the external structure and a base attachment part configured to fix movement of the base component in a first direction and allow radial movement of the base component around the base attachment part.

Some examples of the method further include attaching the coupling component to the external structure using the structure attachment part.

Some examples of the method further include providing a second base component that is connected to the base component via one or more interior base components. Some examples further include attaching the second base component to a second coupling component, where the second coupling component is configured to connect to the external structure to restrict the radial movement of the base component.

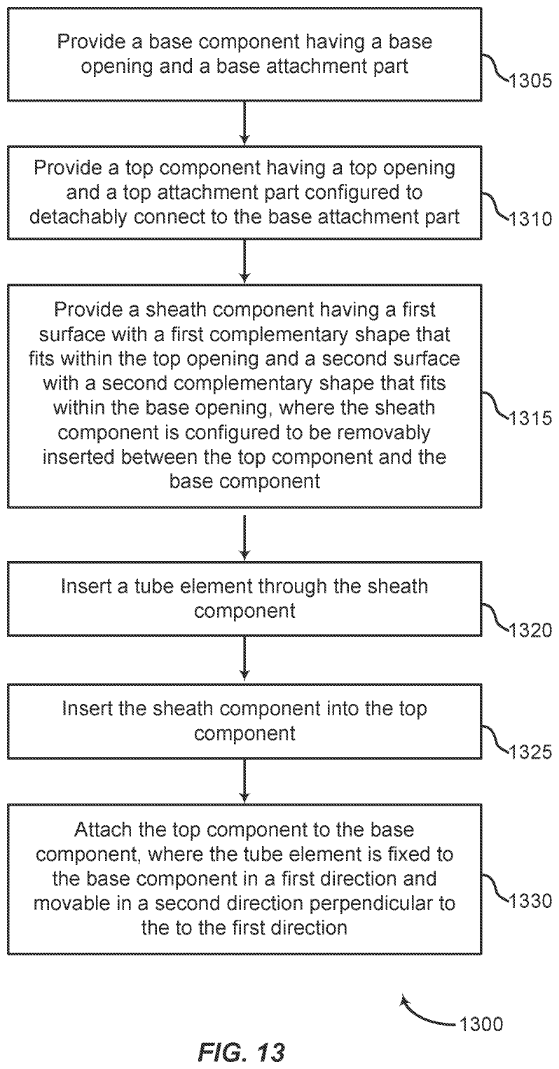

FIG. 13 shows an example of a method 1300 for medical procedures according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

In some cases, various aspects of operations 1305-1330 may be performed by, or facilitated by a medical professional (e.g., as described in more detail herein).

At operation 1305, the system provides a base component having a base opening and a base attachment part. In some cases, the operations of this step refer to, or may be performed by, a base component as described with reference to FIGS. 3-6, 10, and 11.

At operation 1310, the system provides a top component having a top opening and a top attachment part configured to detachably connect to the base attachment part. In some cases, the operations of this step refer to, or may be performed by, a top component as described with reference to FIGS. 3, 5, 6, and 8-11.

At operation 1315, the system provides a sheath component having a first surface with a first complementary shape that fits within the top opening and a second surface with a second complementary shape that fits within the base opening, where the sheath component is configured to be removably inserted between the top component and the base component.

In some cases, the operations of this step refer to, or may be performed by, a sheath component as described with reference to FIGS. 7-11.

At operation 1320, the system inserts a tube element through the sheath component. In some cases, the operations of this step refer to, or may be performed by, a tube element as described with reference to FIGS. 1, 2, and 7-11. In some cases, the operations of this step refer to, or may be performed by, a sheath component as described with reference to FIGS. 7-11.

At operation 1325, the system inserts the sheath component into the top component. In some cases, the operations of this step refer to, or may be performed by, a sheath component as described with reference to FIGS. 7-11. In some cases, the operations of this step refer to, or may be performed by, a top component as described with reference to FIGS. 3, 5, 6, and 9-11.

At operation 1330, the system attaches the top component to the base component, where the tube element is fixed to the base component in a first direction and movable in a second direction perpendicular to the to the first direction. In some cases, the operations of this step refer to, or may be performed by, a top component as described with reference to FIGS. 3, 5, 6, and 9-11. In some cases, the operations of this step refer to, or may be performed by, a base component as described with reference to FIGS. 3-6, 10, and 11.

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

The described methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise random access memory (RAM), read-only memory (ROM), electrically erasable programmable read-only memory (EEPROM), compact disk (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

What is claimed is:

1. Apparatus for medical procedures, comprising:
a base component having a base opening and a base attachment part;
a top component having a top opening and a top attachment part configured to detachably connect to the base attachment part; and
a sheath component having a first surface with a first complementary shape that fits within the top opening and a second surface with a second complementary shape that fits within the base opening, wherein the sheath component is configured to be removably inserted between the top component and the base component.

2. The apparatus of claim 1, further comprising:
a plurality of additional base components, wherein one of the plurality of additional base components is detachably connected to the base component and each of the plurality of additional base components is detachably connected to another of the plurality of additional base components.

3. The apparatus of claim 2, further comprising:
a plurality of additional top components and a plurality of additional sheath components, wherein each of the plurality of additional top components and each of the plurality of additional sheath components corresponds to an additional base component of the plurality of additional base components.

4. The apparatus of claim 3, wherein:
each of the plurality of additional sheath components comprises a sheath opening having a same size and shape as the sheath component.

5. The apparatus of claim 3, wherein:
at least one of the plurality of additional sheath components comprises a sheath opening having a different shape from the sheath component.

6. The apparatus of claim 1, wherein:
the base component comprises an upper surface having a half-pipe shape corresponding to a bottom surface of the sheath component.

7. The apparatus of claim 1, further comprising:
a coupling component connected to the base component, wherein the coupling component comprises a structure attachment part configured to connect the coupling component to an external structure and a base attachment part configured to connect the coupling component to the base component.

8. The apparatus of claim 7, wherein:
the base attachment part comprises a snap hook that is detachable from the base component.

9. The apparatus of claim 1, wherein:
the base component, the top component, and the sheath component are formed of a same material.

10. The apparatus of claim 1, wherein:
the top attachment part comprises an extension part that is configured to be inserted into a slot of the base attachment part with a rotational motion of the top attachment part around a central axis of the top opening.

11. The apparatus of claim 1, wherein:
the sheath component has a circular opening configured to wrap around a tube element and affix the tube element to the apparatus.

12. The apparatus of claim 11, wherein:
the same material comprises a rigid plastic material.

13. The apparatus of claim 1, wherein:
the sheath component comprises a first half-pipe part and a second half-pipe part that is detachably connected to the first half-pipe part.

14. Apparatus for medical procedures, comprising:
a plurality of base components, wherein each of the plurality of base components has a base attachment part;
a plurality of top components corresponding to the plurality of base components, wherein each of the plurality of top components has a top opening and a top attachment part configured to detachably connect to a corresponding base attachment part; and
a plurality of sheath components, wherein each of the plurality of sheath components has an outer surface having a same shape as the top opening of a corresponding top component and is configured to be removably inserted into a corresponding top component.

15. The apparatus of claim 14, further comprising:
a coupling component connected to a base component of the plurality of base components, wherein the coupling component comprises a structure attachment part configured to fix a position of the coupling component relative to an external structure and a base attachment part configured to fix movement of the plurality of base components in a first direction and allow movement of the plurality of base components in a radial motion around the base attachment part.

16. The apparatus of claim 14, wherein:
each of the plurality of base components comprises a bottom surface that is parallel to a bottom surface of each of the other base components of the plurality of base components.

17. Method for medical procedures, comprising:
providing a base component having a base opening and a base attachment part;
providing a top component having a top opening and a top attachment part configured to detachably connect to the base attachment part; and
providing a sheath component having a first surface with a first complementary shape that fits within the top opening and a second surface with a second complementary shape that fits within the base opening, wherein the sheath component is configured to be removably inserted between the top component and the base component.

18. The method of claim 17, further comprising:
inserting a tube element through the sheath component;
inserting the sheath component into the top component; and
attaching the top component to the base component, wherein the tube element is fixed to the base component in a first direction and movable in a second direction perpendicular to the to the first direction.

19. The method of claim 17, further comprising:
attaching the base component to a coupling component, wherein the coupling component comprises a structure attachment part configured to extend around an edge of an external structure to fix a position of the coupling component relative to the external structure and a base attachment part configured to fix movement of the base component in a first direction and allow radial movement of the base component around the base attachment part.

20. The method of claim 19, further comprising:
providing a second base component that is connected to the base component via one or more interior base components; and
attaching the second base component to a second coupling component, wherein the second coupling component is configured to connect to the external structure to restrict the radial movement of the base component.
